(19) United States
(12) Patent Application Publication
(10) Pub. No.: US 2011/0060039 A1
(43) Pub. Date: Mar. 10, 2011

(54) COMPOSITION

(76) Inventors: Herwig Bernaert, Lebbeke-Wieze (BE); Leen Allegaert, Lebbeke-Wieze (BE)

(21) Appl. No.: 12/864,353

(22) PCT Filed: Jan. 23, 2009
(86) PCT No.: PCT/EP2009/000443

§ 371 Date: Aug. 25, 2010

(30) Foreign Application Priority Data
Jan. 25, 2008 (GB) 0801401.1

(51) Int. Cl. A61K 8/49; A61Q 19/00

(52) U.S. Cl. 514/456

(57) ABSTRACT

A cocoa powder having a polyphenol content of at least 5% by weight, may be used to provide skin benefits by oral administration. Preferably, the cocoa powder is provided as part of a foodstuff or confectionary product, or as a cosmetic composition or supplement, The skin benefit preferably comprises a reduction in the degree of wrinkling of the skin and/or an improvement in the smoothness of the skin.

This invention relates to uses of a cocoa powder. In particular, the invention relates to uses of a cocoa powder for skin benefits.

Chocolate and cocoa are popularly claimed to have a plethora of positive effects, including stimulant, relaxant, euphoriant, aphrodisiac, tonic and antidepressant properties. However, the scientific basis for these claims has been elusive. Certainly, depression may in some individuals lead to a craving for sweet foods, and people may receive a transitory uplift in mood from the pleasure of consuming chocolate or from relief of hypoglycemia due to consumption of the sugar in the chocolate. However, the various chemicals in chocolate (other than sugar) suggested to have potentially psychoactive or mood altering effects are generally not present at pharmacologically effective levels.

Traditionally, cocoa for the production of chocolate is made from the dried and partially fermented seeds of the cacao tree. The harvested cacao pods are opened, the pulp and cocoa beans are removed, and the rind is discarded. The pulp and beans are then piled in heaps, placed in bins, or laid out on grates for usually around 6 days, during which time the thick pulp liquifies as it ferments. The fermented pulp trickles away, leaving the cocoa beans behind to be collected, dried and further processed to make cocoa butter and cocoa powder. In some instances, the product is treated with alkali to reduce the acidity of the powder. Fermentation is important for the quality and flavor of the beans, which originally have a strong bitter taste. Unfermented or underfermented cocoa beans have a flavour similar to raw potatoes, are very susceptible to mildew and fungal growth, and therefore are not used in the manufacture of chocolate for food consumption. The cocoa bean without its shell is known as a “cocoa nib”.

Cocoa is known to contain polyphenols and other biologically active compounds such as xanthines, including theobromine and caffeine.

It is known that fresh cocoa beans contains approximately 40% water, 30 to 35% lipids, 4 to 6% polyphenols or polyphenol derivatives, 1.5% xanthines, with the rest being mainly constituted by proteins, starch, cellulose and sugars. Further information on the composition of cocoa beans may be found in the following articles:

Cocoa procyandins: major flavanoids and identification of some minor metabolites by L. J. Porter, Z. Ma and B. G. Chan, published in Phytochemistry vol. 35, No. 5 p 1657-1663, 1991 and

Epicatechin content in fermented and unfermented cocoa beans by H. Kim and P. G. Keeney, published in Journal of Food Science-vol. 49 (1984) p 1090-1092.

Cocoa is grown in South America, Africa and elsewhere. When harvested, its fruits or pods are picked, and the beans undergo a pre-treatment which consists of fermenting for five to six days before being dried. During this fermentation, a certain number of biochemical reactions occur, which involve, in particular, the destruction of pathogenic micro-organisms, the formation of aroma precursors and a partial degradation of polyphenols following enzymatic oxidation or tanning of proteins. It is considered that 70 to 80% of polyphenols are degraded during traditional fermentation.

Polyphenols are a diverse group of compounds (Ferriera et al., “Diversity of Structure and Function in Oligomeric Flavanoids, Tetrahedron, 48:10, 1743-1803, 1992). They occur widely in a variety of plants, some of which enter into the food chain. Several thousands of molecules with a polyphenol structure have been identified in higher plants, and several hundreds are found in edible plants. These molecules are secondary metabolites of plants and generally involved in defense against external stressors, like ultraviolet radiation or aggression by pathogens (Manach, C., et al., Polyphenols: food sources and bioavailability. Am J Clin Nutr, 2004. 79: p. 727-47).

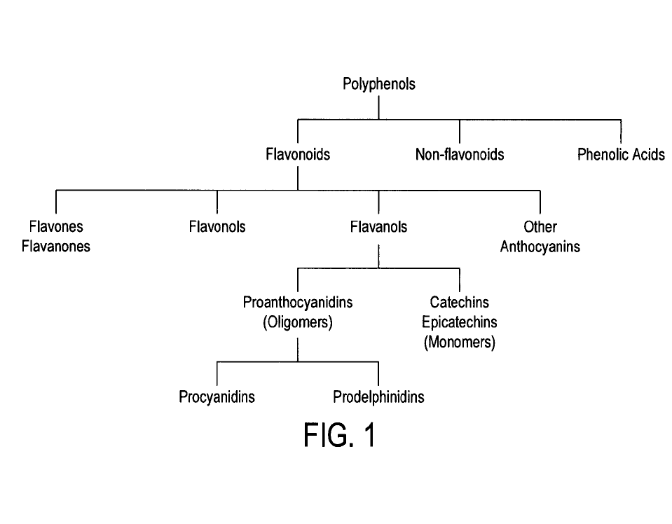

Polyphenols may be further classified into different groups as a function of the number of aromatic rings and the structural elements that bind these rings together. Distinctions are made between flavonoids, non-flavonoids and phenolic acids (see FIG. 1), with the flavanoids being the largest group with more than 2000 known compounds. FIG. 1 shows the classification hierarchy of polyphenols with epicatechin, catechin and the procyanidins being the predominant class of polyphenols in cocoa.

Cocoa and cocoa-derived products are rich in polyphenols and particularly in flavonoids, a class of compounds that occur in a wide variety of fruits, vegetables, teas and red wines. It is well documented that cocoa and cocoa products, like chocolate, are among the richest sources of polyphenols (Ding, E., et al., Chocolate and prevention of cardiovascular disease: a systemic review. Nutr & Met, 2006, 3:1-12).

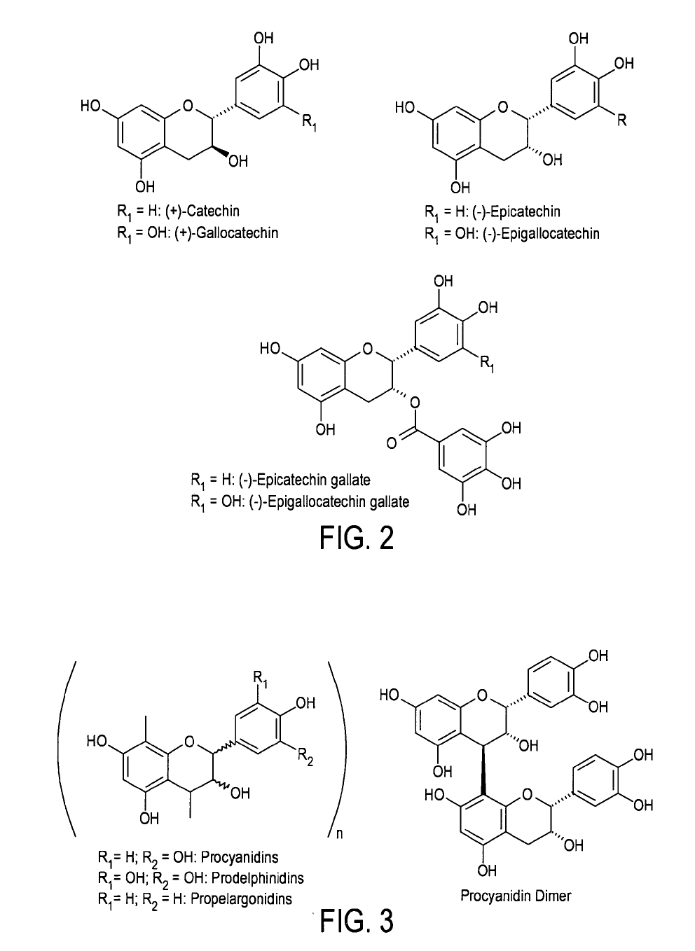

In addition, cocoa has been described as being rich in a particular subgroup of flavonoids named flavanols (flavan-3-ols). The flavanols are present as the monomers epicatechin and catechin or as oligomers of epicatechin and/or catechin called procyanidins. The chemical structures of the different flavanols found in cocoa are shown in FIGS. 2 and 3. FIG. 2 shows the chemical structure of the flavanol monomers in cocoa. FIG. 3 shows the chemical structure of flavanol dimers and oligomers in cocoa.

An increasing body of evidence supports the concept that dietary intake of polyphenols promotes health and attenuates or delays the onset of various diseases, including cardiovascular diseases, cancer, and other chronic diseases.

Flavanols in cocoa and cocoa products exert some beneficial vascular effects (Schroeter, H., et al., Epicatechin mediates beneficial effects of flavanol-rich cocoa on vascular function in humans. PNAS, 2006. 103: p. 1024-1029; Engler, M. B., et al., Flavonoid-rich dark chocolate improves endothelial function and increases plasma epicatechin concentrations in healthy adults. Journal of the American College of Nutrition, 2004. 23(3): p. 197-204).

Flavanols in cocoa and cocoa products reduce the risk for cardiovascular morbidity and mortality (Buijsse, B., et al., Cocoa intake, blood pressure and cardiovasular mortality. The Zutphen Elderly Study. Arch Intern Med, 2006. 166: p. 411-417). Flavanols in cocoa and cocoa products also reduce the risk for cancer (Yamagishi, M., et al., Chemoprevention of lung carcinogenesis by cacao liquor proanthocyanidins in a male rat multi-organ carcinogenesis model. Cancer letters, 2003. 191: p. 49-57). Flavanols in cocoa and cocoa products may contribute to the prevention of neurodegenerative diseases and diabetes mellitus (Bayard, V., et al., Does flavanol intake influence mortality from nitric oxide-dependent processes? Ischemic heart disease, stroke, diabetes mellitus, and cancer in Panama. Int. J. Med. Sci., 2007. 4(1): p. 53-58).

A research group found that long-term ingestion of cocoa flavanols photoprotection against UV-induced erythema and improves skin condition in women (Heinrich, U., et al J. Nutr., 2006. 136: p. 1565-1569). In another study from the same group, an increase in dermal blood flow and oxygen saturation of haemoglobin was detected within two hours after ingestion of a single-dose of flavanol-rich cocoa (Neukam, K., et al., Consumption of flavanol-rich cocoa acutely increases microcirculation in human skin. Eur J Nutr, 2007. 46:1, p. 43-56).

Polyphenols are powerful natural anti-oxidant and anti-radical substances. Polyphenol extracts and preparations which contain them are usually used in the following indications: circulatory disorders, venous-lymphatic insufficiency, cutaneous capillary fragility, retinal circulatory disorders, haemorrhoids, rashes caused by the sun or associated with the effect of radiation (prevention of damage caused by radiotherapy), hypertension, hypercholesterolemia, various viral and microbial illnesses. Numerous publications have revealed types of action at a molecular level by which they are capable of fighting major illnesses including:

### Cardiovascular Diseases:

Platelet antiaggregates (Petroni, A., M. Blasevich, M. Salami, N. Papini, G. F. Montedoro and C. Galli, “Inhibition of platelet aggregation and eicosanoid production by phenolic components of olive oil”. Thromb Res, 1995. 78(2): p. 151-160)

Anti-inflammatories and protection against the oxidation of LDL-cholesterols (Frankel, E., J. Kanner, J. German, E. Parks and J. Kinsella, “Inhibition of oxidation of human low-density lipoprotein by phenolic substances in red wine”. Lancet, 1993. 341(8843): p. 454-457).

Protection against the oxidation of eicosanoids (Pace-Asciak, C. R., S. Hahn, E. P. Diamandis, G. Soleas and D. M. Goldberg, “The red wine phenolics transresveratrol and quercetin block human platelet aggregation and eicosanoid synthesis: implications for protection against coronary heart disease”. Clin Chim Acta, 1995. 235(2): p. 207-219)

Anti-atheroscierotics (Yamakoshi, J., S. Kataoka, T. Koga and T. Ariga, “Proanthocyanidin-rich extract from grape seeds attenuates the development of aortic atherosclerosis in cholesterol-fed rabbits”. Atherosclerosis, 1999. 142(1): p. 139-149)

Anti-thrombotics (Fuhrman, B., A. Lavy and M. Aviram, “Consumption of red wine with meals reduces the susceptibility of human plasma and low-density lipoprotein to lipid peroxidation”. Am J Clin Nutr, 1995. 61(3): p. 549-554)

Alzheimers (Orgogozo, J. M., J. F. Dartigues, S. Lafont, L. Letenneur, D. Commenges, R. Salamon, S. Renaud and M. Breteler, “Wine consumption and dementia in the elderly: A prospective community study in the Bordeaux area”. Rev Neurol, 1997. 153(3): p. 185-192)

Cancer (Jang, M. S., E. N. Cai, G. O. Udeani, K. V. Slowing, C. F. Thomas, C. W. W. Beecher, H. H. S. Fong, N. R. Farnsworth, A. D. Kinghorn, R. G. Mehta, R. C. Moon and J. M. Pezzuto, “Cancer chemopreventive activity of resveratrol, a natural product derived from grapes”. Science, 1997. 275(5297): p. 218-220)

Taking into account the fact that cocoa contains polyphenols and of the importance of the use of polyphenols in the medical field, this has led to an attempt to extract the polyphenol compounds from cocoa, with the aim of creating dietetic food and drinks containing these antioxidants. The traditional pre-treatment, comprising fermentation followed by a drying operation, constitutes a major drawback in the sense that it reduces the levels of polyphenols contained in the resulting cocoa products.

US 2004/096566 discloses a process for carrying out the extraction under specific conditions that makes it possible to process cocoa beans to provide products with a high polyphenol content and enriched, (in comparison to the initial content of the beans) with certain useful lipid derivatives. The process disclosed in US 2004/096566 comprises the use of fresh beans, not having undergone a pre-treatment or defatting, these beans having had their pulp and shell removed, in such a way as to obtain clean kernels, the grinding of said kernels in the presence of a solvent, the maceration of the ground kernels under conditions allowing the desired compounds to be extracted, the filtration of the maceration mixture, and the recovery of the extract containing said compounds from the filtrate.

US 2007/0258920 discloses a further process for the production of cocoa based materials having enhanced levels of polyphenols.

WO2007/002883 discloses products, including beverages, that contain polyphenols and sterol and/or stanol esters, that are produced by a method which conserves the levels of polyphenols found in the starting materials. This documents teaches that the acidification of polyphenol-containing ingredients helps to retain their levels of polyphenols during their incorporation into the final products. The disclosed method is based on combining all the ingredients of the product together and thereafter adding acid to reduce the pH before further processing.

Cocoa extracts containing polyphenols have been proposed for a number of uses. For example, WO 96/010404 describes cocoa extracts containing proanthocyanidins that are said to be anti-neoplastic. U.S. Pat. No. 7,122,574 discloses polyphenol-containing cocoa extracts that can be used for treating hypertension. WO 03/079998 states that cocoa extracts containing polyphenols can be used in the treatment of diseases involving defective gap junctional communication.

Actives in cocoa extracts other than polyphenols have also been used in an attempt to achieve physiological effects. For example, U.S. Pat. No. 6,927,280 discloses a cocoa albumin and its uses. U.S. Pat. No. 7,115,285 relates to a composition, comprising theobromine or a salt thereof, for suppressing appetite and cravings for substances such as nicotine, coffee, sweets or chocolate while improving energy and enhancing mood. WO 2007/042745 discloses a composition comprising chocolate which is enhanced with theobromine and reviews the active components in chocolate, stating that cocoa contains a number of chemical substances whose influence on human and/or animal physiology is not fully understood, including phenylethylamine and tyramine which act as neurotransmitters and may effect mood swing by causing an emotional high, which can be associated with a feeling of alertness and contentment.

US20070148107 describes a method of reducing UV-induced skin erythema and/or photoaging in a subject in need thereof comprising orally administering to the subject a composition comprising an effective amount of a cocoa component.

FR2885050A1 discloses a slimming cosmetic and/or pharmaceutical composition for the treatment of the adipocytes of skin which comprises a cocoa extract containing polyphenols. There is no mention of exactly how the cocoa extract is obtained.

US20060134179 relates to a health food product comprising proanthocyanidins, ascorbic acid or a derivative thereof, and L-cysteine or a derivative thereof. The product is said to provide an excellent beautification (skin-beautifying) effect.

WO 02/14251 describes a method for obtaining cocoa bean polyphenol extracts by solvent extraction of fresh cocoa beans. The extracts have cosmetic, food and therapeutic uses and may contain increased levels of beta-sitosterol.

WO 2007/082703 relates to the use of cocoa polyphenols, which may be produced by the method described in WO 02/14251, in beer production.

There remains a need for orally administrable compositions that are useful for providing skin benefits, particularly compositions that are derived from natural products. There also remains a need for compositions having these benefits that can be readily incorporated into formulations for oral consumption. For example, the compositions for incorporation into foods and beverages are desirably readily dispersible and impart a good appearance to the product, in terms of colour and/or texture.

According to the invention, there is provided a cocoa powder having a polyphenol content of at least 5% by weight, for use in providing a skin benefit by oral administration.

Preferably, the cocoa powder has a polyphenol content of from 5 to 25% by weight.

Conveniently, the cocoa powder has a polyphenol content of from 7 to 15% by weight.

Advantageously, the cocoa powder has a polyphenol content of from 8 to 12% by weight.

Preferably, the cocoa powder comprises at least 5 mg/g of epicatechin.

Conveniently, the cocoa powder comprises at least 10 mg/g of epicatechin.

Advantageously, the cocoa powder comprises from 15 to 25 mg/g of epicatechin.

Preferably, the cocoa powder comprises at least 1.5 mg/g catechin.

Conveniently, the cocoa powder comprises at least 2.5 mg/g catechin.

Advantageously, the cocoa powder comprises from 3 to 6 mg/g catechin.

Preferably, the cocoa powder has an ORAC value of greater than 1000 micromoles TE/g.

Conveniently, the cocoa powder has an ORAC value of greater than 1500 micromoles TE/g.

Advantageously, the cocoa powder is provided as part of a foodstuff or confectionery product.

Preferably, the foodstuff is a beverage.

Conveniently, the cocoa powder is provided as a cosmetic composition or supplement.

According to a further aspect of the invention, there is provided the use of a cocoa powder having a polyphenol content of at least 5% by weight, in the manufacture of a medicament for use in providing a skin benefit by oral administration.

According to another aspect of the invention, there is provided the use of a cocoa powder having a polyphenol content of at least 5% by weight, in the manufacture of a foodstuff for use in providing skin benefits by oral administration.

Preferably, the cocoa powder used is as defined above.

According to a further aspect of the invention, there is provided a method for providing a skin benefit in a subject, comprising orally administering to said subject an effective amount of a cocoa powder having a polyphenol content of at least 5% by weight.

Conveniently, the skin benefit comprises a reduction in the degree of wrinkling of the skin.

Advantageously, the skin benefit comprise an improvement in the smoothness of the skin.

Preferably, the skin benefit does not include reducing UV-induced skin erythema and/or photoaging.

The present invention will now be described, by way of example, with reference to the accompanying figures, in which:

FIG. 1 is a schematic summary of the types of polyphenols that may be present in cocoa powder;

FIG. 2 shows the chemical structure of the cocoa polyphenols catechin, epicatechin and derivatives thereof;

FIG. 3 shows the chemical structure of cocoa procyanidin polyphenols;

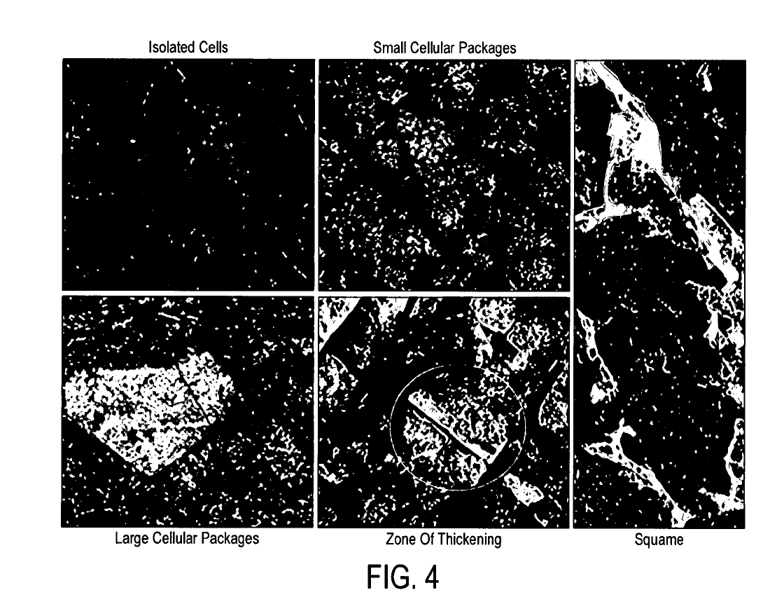

FIG. 4 shows photomicrographs of skin samples taken with D-Squame adhesive tape strippings;

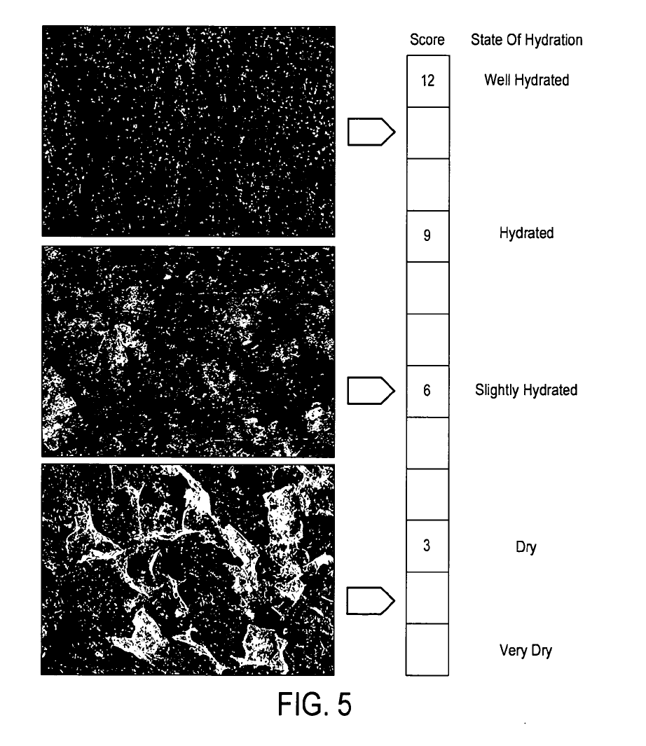

FIG. 5 shows the Diagnoskin® scoring system for evaluation of the state of hydration of skin;

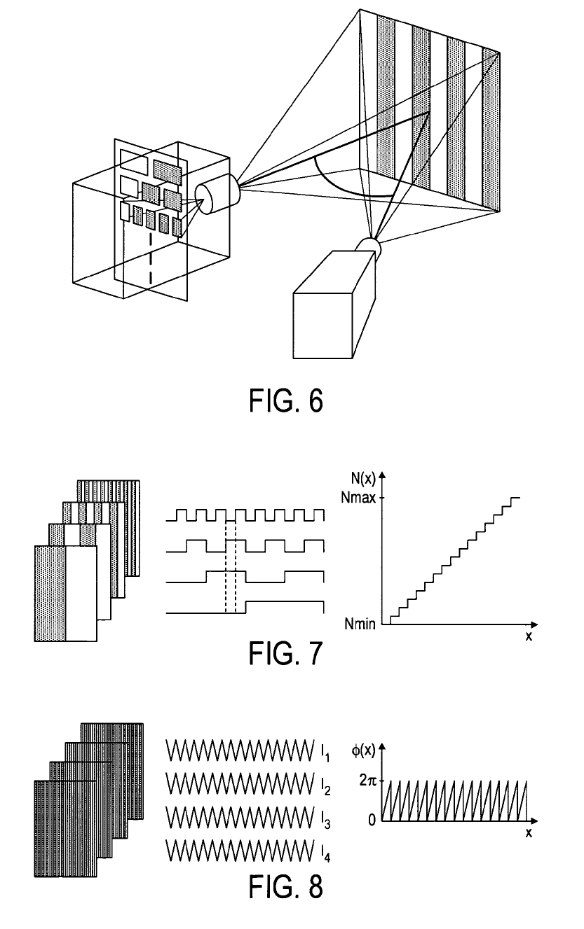

FIG. 6 is a schematic illustration of the use of a video camera in an interference fringe projection measurement technique;

FIG. 7 shows the use of grey-code projection in an interference fringe projection measurement technique; and

FIG. 8 shows the use of phase-shift in an interference fringe projection measurement technique.

It has been found that cocoa powders having a polyphenol level of at least 5% by weight have advantages in terms of their effect on skin after oral consumption. The effects, which are systemic rather than topical, are very different from that achieved by the topical application of cocoa based materials.

The cocoa powder used in the invention has a polyphenol content of at least 5% by weight, such as at least 7% by weight. The upper limit for the polyphenol content is typically about 25% by weight. Thus, preferred amounts of polyphenol include from 5% to 25%, from 7% to 15%, from 8% to 12%, from 9% to 11% and about 10%, the percentages being by weight of the cocoa powder.

In this specification, “cocoa powder” is meant to refer to a single cocoa powder and also, where appropriate, a mixture of two or more cocoa powders. Thus, a cocoa powder having, for example, at least 5% by weight polyphenol content may be a single cocoa powder having at least 5% by weight polyphenol content, or it may refer to mixture of cocoa powders where the mixture has on average at least 5% by weight polyphenol content. Such a mixture may comprise, for example, a cocoa powder having more than 5% by weight polyphenol content and also a cocoa powder having less than 5% by weight polyphenol content, giving a mixture with at least 5% by weight polyphenol content overall

The percentages of polyphenols are preferably expressed as gallic acid equivalents, according to the Folin-Ciocalteu method (e.g., as described in Singleton V L, Orthofer R, Lamuela-Raventos R M. Analysis of total phenols and other oxidation substrates and antioxidants by means of Folin-Ciocalteu reagent. Meth Enzymol 1999; 99: 152-178).

Polyphenols in the cocoa powders used in the invention typically comprise monomers and oligomers. Preferably, the cocoa powders used in the invention comprise up to 20% by weight of each of monomers, dimers, trimers, tetramers, pentamers, hexamers, heptamers, octamers, nonamers and decamers, and higher oligomers in an amount of up to 25% by weight. More preferably, the cocoa powders used in the invention comprise, by weight of the cocoa powder, 15 to 20% monomers (preferably including at least 15% epicatechin), 10 to 15% dimers, 7 to 12% trimers, 6 to 10% tetramers, 6 to 10% pentamers, 6 to 10% hexamers, 2 to 6% heptamers, 2 to 6% octamers, 2 to 6% nonamers and 2 to 6% decamers, and 10 to 25% higher oligmers.

The cocoa powders for use in the invention may contain xanthines (preferably methylxanthines), such as caffeine and theobromine. Caffeine may be present together with theobromine, typically at a weight ratio of theobromine to caffeine in the range of from 20:1 to 5:1. In one embodiment of the invention, the theobromine content is at least 1% by weight, and preferably from 1 to 3% by weight. In this embodiment, the composition preferably has a weight ratio of from 4:1 to 8:1 polyphenol:theobromine. In an alternative embodiment, the cocoa powder may be treated, for example with supercritical carbon dioxide with or without a combination of solvent and pressure, to lower the theobromine content and the content of other xanthines that may be present. A method for lowering the content of theobromine in cocoa materials is described in Example 2.3 of WO 2007/082703, the contents of which are incorporated herein by reference. In this alternative embodiment, the cocoa powder has a theobromine content of less than 2% by weight, such as less than 1.5% by weight, for example from 0.1 to 1.5% by weight.

By “polyphenol” we mean the well-known group of chemical substances that are found in plants, characterised by the presence more than one phenol group per molecule. Polyphenols are often present as monomers, dimers, trimers and other oligomers. Flavonoids are a subset of polyphenol. Cocoa contains polyphenols such as catechin, epicatechin, gallocatechin, epigallocatechin, epicatechin gallate, epigallocatechin gallate, procyanidins, prodelphinidins, and propelargonidine. Preferred polyphenols include procyanidins A2, B1 to B5, and C-1. Polyphenols with a molecular weight of less than 3000 are preferred.

The cocoa powders for use in the invention have an enhanced level of polyphenols. By “enhanced levels of polyphenols” it is meant that the cocoa based material contains a level of polyphenols that is higher than that found in traditionally prepared cocoa based materials. One example of traditionally prepared cocoa based material is alkalized cocoa powder. Thus, a cocoa powder that contains more polyphenols than an alkalized cocoa powder has an enhanced level of polyphenols. A cocoa powder that contains an enhanced level of polyphenols may be prepared using a controlled cocoa bean fermentation process. More specifically, a cocoa powder having an enhanced level of polyphenols contains at least 50 mg of polyphenols per gram of cocoa powder (50 mg/g or 5% by weight).

An alkalised cocoa powder will generally contain no more than about 3.5% by weight polyphenols. A natural cocoa powder will generally contain more polyphenols than an alkalised cocoa powder. However, natural cocoa powders have a relatively low pH of around 5.5 which can cause handling problems. Preferred cocoa based materials having enhanced levels of polyphenols are those with a pH greater than that of natural cocoa powder, more preferably having a pH of greater than about 6, most preferably having a pH of between about 6.2 and about 6.4.

Preferably, the foodstuffs of the invention including beverages, do not contain any added alcohol. Preferred drinks of the invention are substantially free from alcohol.

The cocoa based material used in the invention has an enhanced level of polyphenols and is preferably derived from cocoa beans using a process designed to retain the polyphenols present in the harvested cocoa beans.

One well known way to determine the level of polyphenols in a product is to measure its ORAC value. ORAC stands for Oxygen Radical Absorbance Capacity and is a measurement for the capacity of a sample to neutralize free radicals. This capacity can be measured using a test method known as the ORAC assay.

This test uses AAPH as a physiological relevant radical generator and fluorescein as a fluorescent probe. As an internal standard in this assay, Trolox (a water-soluble analogue of vitamin E) is used. Fluorescein is an intense and long lasting-fluorescent probe. Reaction of AAPH with fluorescein leads to a loss of fluorescence. When a sample of material has antioxidative capacities, it neutralizes/scavenges the AAPH radicals and thereby protects the fluorescein from losing its fluorescence. This antioxidant capacity of a sample of material, as measured using the ORAC assay, is called the ORAC value. Since Trolox is used as an internal standard, this ORAC value is expressed as Trolox Equivalents or TE. The international standard way of expressing an ORAC value is micromol TE/mg (or mL) of sample. For particular applications, ORAC values may be converted to other units such as TE/Liter or TE/serving. As used herein, the ORAC value is a value indicating the antioxidant capacity of a sample of material as expressed in units of micromoles Trolox Equivalents (TE) per gram of material (micromoles TE/g).

Preferably, the cocoa powder used in the present invention has an ORAC value of over 1,000 micromoles TE/g, more preferably above 1,500 micromoles TE/g.

One preferred cocoa powder having an enhanced level of polyphenols is available from the Barry Callebaut group under the brand name ACTICOA™. A sample of ACTICOA™ cocoa powder has been measured to have an ORAC value of 2129 micromoles TE/g. This is higher than a measured ORAC value of natural cocoa powder of 826 micromoles TE/g, which itself is higher than the level for traditionally prepared alkalized cocoa powder, of which a sample had a measured ORAC value of 402 micromoles TE/g. Also, ACTICOA™ cocoa powder has a pH of around 6.3.

Cocoa powders used in the invention preferably comprise less than 2% by weight phenylethylamine.

The cocoa powders are used in the invention for providing skin benefits (preferably in a human). The term “skin benefits” is used herein to refer to one or more desirable effects in skin, including general improvements in skin health. Skin benefits include, for example, one or more of: increased firmness, increased elasticity, increased tonicity, reduced wrinkles (including wrinkle width and/or volume), reduced fine lines, increased hydration, decreased skin roughness, decreased scaling, improved skin smoothness, improved skin structure (including skin barrier). The skin benefits most preferably include reduced wrinkles (including wrinkle width and/or volume), and/or improved skin smoothness. Preferably, the skin benefits do not comprise reducing UV-induced skin erythema and/or photoaging.

The cocoa powders used in the invention are formulated for oral consumption and are not intended for topical application. The cocoa powders used in the invention are consumed in a form that is edible and non-toxic. For example, the cocoa powder may be provided as part of a foodstuff or confectionery product. Typically, the cocoa powder will be included in the foodstuff or confectionery product in an amount of from 0.1% to 80% by weight, such as from 0.5% to 40% by weight.

Foodstuffs and confectionery products include, for example, those having a fat continuous phase as well as those having a water continuous phase. Foodstuffs include foods and beverages.

Beverages include those adapted for consumption hot or cold. Beverages may include one or more additives selected from sweeteners, flavouring agents, colouring agents, stabilisers and preservatives. Beverages will typically comprise from 50% to 99% water. Beverages will typically comprise the cocoa powders dispersed and/or suspended therein. The cocoa powder used in the invention is preferably incorporated into the beverage in an amount of from 0.1 to 10% by weight. The cocoa powder may be formulated as a powder which can be converted to a beverage on the addition of water and mixing.

A particularly preferred beverage is a cocoa-flavoured drink. Cocoa-flavoured drinks comprise a: cocoa powder containing at least 5% by weight polyphenols; and optionally milk or a product derived from milk (such as skimmed milk powder) and/or a sweetener. The cocoa powder may be a full fat or a reduced fat cocoa powder. Sweeteners include sugars and non-saccharide sweeteners. Cocoa-flavoured drinks may be formulated to be consumed hot (such as hot chocolate) or cold (such as milk shake).

Foodstuffs typically comprise one or more of protein, fat and carbohydrate. Foodstuffs include dairy products and confectionery products. A preferred foodstuff comprises vegetable fat and/or cocoa butter. Particularly preferred foodstuffs include chocolate and chocolate-like products comprising cocoa solids and sugar. For example, the cocoa powders for use in the invention may be included in conventional chocolate or chocolate-like products in amounts of from 0.1% to 80% by weight, such as from 0.5% to 40% by weight.

The cocoa powders for use in the invention may be included in chocolate or chocolate-like products. The chocolate or chocolate-like products preferably comprise one or more components selected from the group consisting of cocoa powders, cocoa materials, sugars, sugar substitutes, milk powders, fat, emulsifier, flavouring agents and mixtures thereof. Preferably, the cocoa materials are selected from cocoa mass, cocoa liquor, cocoa butter and mixtures thereof. Milk powders include, for example, skimmed milk powder, whey powder and derivatives thereof, full cream milk powder and mixtures thereof. Suitable sugars include sucrose, fructose, glucose and dextrose and mixtures thereof (with sucrose being preferred). Sugar substitutes preferably include inulin, dextrin, isomaltulose, polydextrose and maltitol and mixtures thereof. Fats include butter fat or fractions thereof, palm oil or fractions thereof, coconut or fractions thereof, palm kernel oil or fractions thereof, liquid oils (for example, sunflower oil and/or rapeseed oil), interesterified mixtures of the above fats or fractions or hardened components thereof, or mixtures thereof. Emulsifiers include lecithin, fractionated lecithin and PGPR or mixtures thereof. Flavouring agents include vanilla and caramel or mixtures thereof.

Chocolate and chocolate-like products may comprise one or more food additives such as biscuit, nuts (whole or pieces), crispies, sponge, wafer or fruit, such as cherries, ginger and raisins or other dried fruit. These additives are normally embedded in the product.

Alternatively, the cocoa powder may be provided as a cosmetic composition or supplement.

Cosmetic compositions are preferably in the form of tablets, pills, capsules, caplets, multiparticulates including: granules, beads, pellets and micro-encapsulated particles; powders, elixirs, syrups, suspensions and solutions. Cosmetic compositions will comprise an acceptable diluent or carrier. Orally administrable compositions may be in solid or liquid form and may take the form of tablets, powders, suspensions and syrups. Optionally, the compositions comprise one or more flavouring and/or colouring agents. Carriers suitable for use in such compositions are well known in the art. The compositions of the invention may contain 0.1 to 99% by weight of the cocoa powder.

Supplements may, for example, comprise the cocoa powder in liquid form (e.g., as a solution, dispersion or suspension) and/or encapsulated in a capsule. Supplements (which term includes dietary and nutritional products) may take the form of a soft gel or a hard capsule comprising an encapsulating material, preferably selected from the group consisting of gelatin, glycerol, starch, modified starch, starch derivatives such as glucose, sucrose, lactose and fructose. The encapsulating material may optionally contain cross-linking or polymerizing agents, stabilizers, antioxidants, light absorbing agents for protecting light-sensitive fills, preservatives and the like. Preferably, the amount of the cocoa powder in the food supplements is from 1 mg to 1000 mg (such as from 50 to 500 mg).

As used herein, the term “effective amount” refers to the amount of a cocoa powder or composition which is effective, upon single or multiple dose administration to a subject for achieving one or more skin benefits. An effective amount of the cocoa powders for use in the invention, is in general, about 0.1 to 20 g/day, e.g., 1 to 10 g/day for an adult human, most preferably from 0.5 to 5 g/day. The daily dose may be administered once per day, or in divided doses.

The term “administering” and related terms used herein includes consumption of a composition or other product and does not necessarily imply the involvement of any medically qualified personnel. The cocoa powders, compositions and other products can, for example, be administered by the subject simply consuming the cocoa powder, composition or other product, such as a food or beverage, after having purchased it his- or herself, independently of any supervision or treatment regimen.

The listing or discussion of an apparently prior-published document in this specification should not necessarily be taken as an acknowledgement that the document is part of the state of the art or is common general knowledge.

The following non-limiting examples illustrate the invention and do not limit its scope in any way. In the examples and throughout this specification, all percentages, parts and ratios are by weight unless indicated otherwise.

### EXAMPLE 1

### I. Summary

### Evaluation of the Bio-Efficiency of a Polyphenol Rich Cocoa Drink on Skin Health Parameters Hydration and Wrinkles.

1 Experimental design: randomised, double-blind, parallel study.
2 Participants: 48 volunteers, aged 18 to 65 years (24 between 18 to 44 years and 24 between 44 to 65 years), males and females.
3 Duration: 12 weeks.
4 Treatments: daily, a high-polyphenol cocoa drink or a low polyphenol cocoa drink.
5 Measurements: anti-wrinkle effect on 24 participants in total (12 high, 12 low), hydration score.
Conclusion: after 12 weeks, the high polyphenol drink was more effective in reducing the width (−1%) and volume (−4%) of the wrinkles compared with the low polyphenol drink (+0.4% and −2.3% for width and volume respectively). There was a larger increase in hydration score in the high polyphenol (+12%) group compared with the low polyphenol group (+7%) after 12 weeks.

### II. Preparation of the Drinks

### Composition of the Dry Powder:

| Ingredients (%) | Low | High |
|---|---|---|
| sugar | 63.3 | 63.3 |
| ACTICOA ™ cocoa powder | 0.0 | 20.0 |
| Alkalized cocoa powder | 33.0 | 13.0 |
| Anti-caking agent (E341) | 0.7 | 0.7 |

### Polyphenol Content of Cocoa Powders

| | ACTICOA ™ | Alkalised |
|---|---|---|
| | cocoa powder | cocoa powder |
| Polyphenols (%, measured | Minimum | Maximum |
| by Folin-Ciocalteu method) | 10% by weight | 3% by weight |

To prepare the drinks, 10 grams of dry powder was poured into 200 cc of drinking water, followed by mixing. One drink was prepared and consumed by all participants every morning during the 12 week trial period.

### III. Methods to Analyse Skin Benefits

### III.A. Hydration

The analysis of surface samples, such as D-Squam® adhesive tape strippings, allows the evaluation of the desquamation's quality. This parameter is correlated with the hydration of the stratum corneum.

A scoring system (from 1 to 12) of the desquamation parameters is used in order to evaluate the state of hydration of the skin (Diagnoskin® system). FIG. 5 shows the scale of 1 to 12 and exemplary photographs of skin samples.

### Background Information

### Hydration Evaluation Technique

Affixing on the skin of a flexible adhesive, D-Squam® retains the surface layer of corneocytes when it is withdrawn. The semiological evaluation of this exfoliation under an optical microscope (objective×25 with cold light) leads to a value from 1 to 12, increasing according to the level of hydration. FIG. 4 shows photographs of typical D-Squam® samples. In this study, this taking away of surface was carried out on the zone of the right front forearm.

### III.B. Wrinkles

The topography of a wrinkled area is studied. A replica of the cutaneous surface is taken with a silicon elastomer (Silflo) on the crow's feet area. Even smallest skin depths are reproduced in detail.

Tridimensional reconstruction is done using interference fringe projection technique. Individual comparative analyse with 3D pictures is then performed. FIGS. 6, 7 and 8 show the principles of this technique.

Analysis of the skin topography may be performed by interference fringe projection technique, associated with a special computer software. The surface of a 3-dimensional object is described by an equation with 3 unknown parameters (x, y, z). The relief of this object is a function f(x,y) describing the altitude z of a point M of the surface z=f(x,y). In order to obtain a certain number of altitude curves, Z=Zi, the object is illuminated with a fringe projection technique (halogen) with an inclined grid, and a field of depth of 6 mm (−3 mm<z<3 mm).

FIGS. 6, 7 and 8 illustrate a suitable system for analysing skin topography using fringe interference techniques, as used in the Dermatop® system from la societéEotech, France. FIG. 6 is a schematic illustration of the use of a video camera in the interference fringe projection measurement technique. FIG. 7 shows the use of grey-code projection in the interference fringe projection measurement technique. FIG. 8 shows the use of phase-shift in the interference fringe projection measurement technique.

### IV. Results

### IV.A. Hydration

Selective group of subjects (18 to 65 years) with low initial hydration score, meaning score 0-6.

0-3: dry skin
3-6: low hydration
n=16/group

| Hydration score | Low | High |
|---|---|---|
| Week 0 | 5.56 | 5.88 |
| Week 12 | 5.94 | 6.56 |
| % Week 12 versus Week 0 | +6.7 | +11.7 |

### IV.B. Wrinkles

n=14 in high and n=11 in Low

| Wrinkles | Low | High |
|---|---|---|
| % in width, Week 12 versus | +0.41 | −0.82 |
| Week 0 | | |
| % in volume, Week 12 | −2.38 | −3.85 |
| versus Week 0 | | |

### EXAMPLE 2

### Evaluation of the Bio-Efficiency of a Polyphenol Rich Cocoa Drink on Skin Hydration

1. Experimental design: Randomized, placebo-controlled, double blind, parallel study.
2. Participants: 32 healthy females (Mid)European, 35 to 65 years, BMI 18 to 27, skin phototype I, II & III (16 persons/group)
3. Duration: 6 weeks.
4. Treatments: daily, flavanol-rich cocoa beverage made with ACTICOA™ cocoa powder providing 500 mg flavanols per day versus a placebo cocoa beverage made with low-flavanol cocoa powder providing 16 mg flavanols per day, 1 portion at breakfast. The cocoa beverages were isocaloric and did not differ in theobromine and caffeine content.

### II. Preparation of the Drinks

To prepare the drinks, 20 grams of dry powder was poured into 250 cc of hot water, followed by mixing. One drink was prepared and consumed by all participants every morning during the 6 week trial period.

The 20 gram vending mix contained 50% sugar and 50% cocoa powder, giving a total of 10 gram of cocoa powder per vending mix. The cocoa powder in each mix consisted of: Placebo: 100% low-flavanol cocoa powder

High Polyphenol Drink: 50% ACTICOA™ cocoa powder and 50% low flavanol cocoa powder

### Composition of the Dry Powder:

| Ingredients (%) | Low | High |
|---|---|---|
| sugar | 50 | 50 |
| ACTICOA ™ cocoa powder | 0 | 25 |
| Alkalized cocoa powder | 50 | 25 |

persachet

| | Placebo | High |
|---|---|---|
| total monomers (mg) | 3.2 | 76.8 |
| total flavanols (mg) | 16 | 500 |
| total theobromine (mg) | 0.26 | 0.25 |
| total caffeine (mg) | 0.018 | 0.022 |

### Polyphenol Content of Cocoa Powders:

| | ACTICOA ™ cocoa | Alkalised cocoa |
|---|---|---|
| | powder | powder |
| Polyphenols (%, measured by | Minimum 10% by | Maximum 1% by |
| Folin-Ciocalteu method) | weight | weight |

10 grams cocoa powder as in ‘low’ drink contains 1% polyphenols (measured by Folin-Ciocalteu method), and in total 12650 ORAC (or 1265 ORAC/g cocoa powder). 10 grams cocoa powder as in ‘high’ drink contains 5.5% polyphenols (measured by Folin-Ciocalteu method), and in total 4000 ORAC (or 400 ORAC/g cocoa powder).

### Hydration:

Skin hydration was measured using the Corneometer™ CM 825 (Courage and Khazaka). Skin hydration measurements depend on the water-holding capacities of the stratum corneum. Dry stratum corneum shows a weak electrical conduction. Hydrated stratum corneum is more sensitive to the electrical field, inducing an increase of dielectric constant. Increasing hydration of the stratum corneum induces an increase in capacitance. The measuring principle of the Corneometer™ CM 825 is based on capacitance measurement. Any change in the dielectric constant due to skin surface capacitance measurement. Any change in the dielectric constant due to skin surface hydration variation alters the capacitance of a precision measuring capacitor. The measurement can detect even slightest changes in the hydration level. The reproducibility of the measurement is very high and the measurement time is very short (˜one second).

### Results:

Hydration (measurement on forearm after 6 weeks):
placebo: −0.3%
High: +8.4% (P=0.07)

1. A cocoa powder having a polyphenol content of at least 5% by weight, for use in providing a skin benefit by oral administration.

2. Cocoa powder as claimed in claim 1 having a polyphenol content of from 5 to 25% by weight.

3. Cocoa powder as claimed in claim 1 having a polyphenol content of from 7 to 15% by weight.

4. Cocoa powder as claimed in claim 1 having a polyphenol content of from 8 to 12% by weight.

5. Cocoa powder as claimed in claim 1 comprising at least 5 mg/g of epicatechin.

6. Cocoa powder as claimed in claim 1 comprising at least 10 mg/g of epicatechin.

7. Cocoa powder as claimed in claim 1 comprising from 15 to 25 mg/g of epicatechin.

8. Cocoa powder as claimed in claim 1 comprising at least 1.5 mg/g catechin.

9. Cocoa powder as claimed in claim 1 comprising at least 2.5 mg/g catechin.

10. Cocoa powder as claimed in claim 1 comprising from 3 to 6 mg/g catechin.

11. Cocoa powder as claimed in claim 1 having an ORAC value of greater than 1000 micromoles TE/g.

12. Cocoa powder as claimed claim 1 having an ORAC value of greater than 1500 micromoles TE/g.

13. Cocoa powder as claimed in claim 1 which is provided as part of a foodstuff or confectionery product.

14. Cocoa powder as claimed in claim 13 wherein the foodstuff is a beverage.

15. Cocoa powder as claimed in claim 1 which is provided as a cosmetic composition or supplement.

16.-18. (canceled)

19. Method for providing a skin benefit in a subject, comprising orally administering to said subject an effective amount of a cocoa powder having a polyphenol content of at least 5% by weight.

20. The method according to claim 19 wherein the skin benefit comprises a reduction in the degree of wrinkling of the skin.

21. The method according to claim 19 wherein the skin benefit comprise an improvement in the smoothness of the skin.

22. The method according to claim 19 wherein the skin benefit does not include reducing UV-induced skin erythema and/or photoaging.
